(19) United States
(12) Patent Application Publication
(10) Pub. No.: US 2012/0052527 A1
(43) Pub. Date: Mar. 1, 2012

(54) LABORATORY METHOD AND EXPERIMENTAL SPECIMEN FOR VALIDATING THE EFFICACY OF ANTIMICROBIAL AGENTS ON BOVINE CARCASSES

(76) Inventors: J. Chance Brooks, Wolfforth, TX (US); Mindy M. Brashears, Wolfforth, TX (US); Gary L. Nace, Claude, TX (US)

(21) Appl. No.: 13/219,503

(22) Filed: Aug. 26, 2011

Related U.S. Application Data
(60) Provisional application No. 61/402,263, filed on Aug. 26, 2010.

(51) Int. Cl. C12Q 1/18

(52) U.S. Cl. 435/32

(57) ABSTRACT

A laboratory method and a test specimen for efficiently and accurately assessing and validating the efficacy of an antimicrobial to treat pathogens on a beef carcass. The method and model employ one or more carcasses from calves that have not received colostrum.

### CROSS-REFERENCE TO RELATED APPLICATION

This application claims the benefit of U.S. Provisional Patent Application Ser. No. 61/402,263, filed Aug. 26, 2010, the disclosure of which is hereby incorporated herein in its entirety by this reference.

### FIELD OF THE INVENTION

The present invention relates to an improved method and specimen for testing the effectiveness of anti-microbial agents in the processing of beef.

### BACKGROUND

Consumer safety is an important issue in the food industry in general and particularly in the industry of supplying animal protein, i.e., edible meat products. By the very nature of animals, the conditions in which they are grown to suitable size, and the nature of the commercial slaughtering process, “meat packers” face serious challenges to ensure that beef products are safe for consumption. When a problem arises in the slaughtering process, the consequences can be serious in terms of public health.—exposing many individuals to potentially serious health consequences, including possibly death. A massive product recall may also cause serious economic consequences to meat producers, processors and others in the distribution chain.

Many of the health issues in the meat industry involve the transmission and growth of microbial pathogens, e.g., Escherichia coli (“E. coli”), Salmonella and other pathogens that can cause sickness and death when ingested by humans. Indeed, Salmonella and another pathogen known as “Campylobacter” are the two leading bacterial causes of food poisoning in the United States. According to the Center for Disease Control, there are 40,000 reported cases of Salmonella poisoning and 600 deaths annually. The CDC estimates that the actual number of Salmonella cases is approximately 30 times the number of reported events. Encountering an immediate bout of illness caused by these pathogens may not be the only consequence. At least one recent report indicates that health effects associated with E. coli and other microbial pathogens may arise months or even years after the initial incident. (“Food Poisoning Legacy: Health Woes can arise Years after Bout, Doctors say,” by Lauran Neergaard reported in The Denver Post, Jan. 22, 2008.) Obviously, it is highly desirable for meat producers to deliver processed meat with minimal incidence of these pathogens.

Despite continuing vigilance for E. coli contamination in beef carcasses and processed beef, E. coli continues to be a recurring problem. Reported cases of sickness and death continue to occur from red-meat contamination, and the industry continues to be at risk to expensive product recalls. These health and economic risks remain despite significant efforts by the industry to avoid them.

To address these concerns, antimicrobial compositions are applied at various stages of beef production. Typically antimicrobial compositions are applied by spraying whole carcasses and spraying or sometimes “bathing” beef “cuts.” Anti-microbial agents, such as peracetic acid, chlorine dioxide and lactic acid may be applied, e.g., typically by spraying, at various points during meat processing. Indeed, the same or different antimicrobial “interventions” are often applied at multiple places as the carcasses move through processing and are disassembled, chilled and packaged for shipment.

Meat packing plants produce a variety of meat cuts, meat products and by-products. After slaughter, skinning, dismemberment and evisceration, a beef carcass is hung from a trolley that enables the carcass to physically move through a series of treatment steps/stations such as chilling and application of an antimicrobial agent. Current practice is to spray the whole carcass with an antimicrobial agent early in production. The carcass is then disassembled into various meat components which may be subjected to additional antimicrobial interventions. Nevertheless, the main focus of antimicrobial intervention is on the carcass where the entire exterior of the animal, i.e., the part most likely to be contaminated, can be treated. Beef producers, the public, and regulatory agencies are concerned with maximizing the effectiveness of this “carcass spray.”

The identification of new antimicrobial agents and the refinement of existing agents are the subject of considerable interest both to improve effectiveness and to lower cost. In addition, the possibility that pathogens such as E. coli and Salmonella become resistant to existing anti-microbial agents emphasizes the need for finding new agents and new modalities of treatment.

Normally, the process of identifying and validating new antimicrobial agents includes both laboratory (i.e., in vitro) and bench work. In each instance, the target is inoculated with a known quantity of pathogen, which is then treated with a known quantity of antimicrobial agent. Effectiveness in treating the pathogen must be adequately demonstrated before government regulators approved and beef producers accept the antimicrobial for commercial use.

It is important, therefore, to be able to efficiently and accurately assess the efficacy of an antimicrobial as an intervention on whole carcasses. At the present time antimicrobial assessments are normally done in the laboratory on beef “cuts.” Evaluation starts by “inoculating” the meat with a known amount of contamination (e.g., E. coli, Salmonella, etc., of an identified strain or strains). The contaminated meat is then treated with a known dose of antimicrobial, and the diminution in the pathogen content is monitored over a period of time. This enables one to assess the effectiveness of the antimicrobial agent against the pathogen.

Food safety considerations preclude the introduction of pathogens onto carcasses in a beef production facility. Accordingly, antimicrobial evaluations of the foregoing type must be performed in an off-site laboratory. The size and weight of beef carcasses make it difficult, if not impossible, to utilize them in a laboratory environment. Thus, smaller, beef “cuts” have been routinely employed.

Unfortunately, the distribution of tissues in a beef cut is not representative of the variety of tissues in a whole carcass. Among other things, cuts tend to contain more lean tissue and less fat. And the fat is not distributed over the surface of the meat as it is in a whole beef carcass. Since the medium (i.e., tissues composition) on which the antimicrobial is tested affects the evaluation of pathogen growth and the effectiveness of antimicrobial intervention, the use of cuts in a laboratory evaluation may yield results that vary significantly from that using the same antimicrobial on an entire carcass. This makes it difficult to get an accurate assessment of the effectiveness of an antimicrobial agent as a beef carcass intervention and to perform comparisons of alternative antimicrobial interventions.

Thus, a new, improved procedure is needed for efficiently and accurately assessing and validating the efficacy of antimicrobials on whole beef carcasses.

### SUMMARY OF THE INVENTION

The invention provides a laboratory method and a test specimen for efficiently and accurately assessing and validating the efficacy of an antimicrobial to treat pathogens on a beef carcass. The method and model employ one or more carcasses from calves that have not received colostrum. In a preferred embodiment the calf also has not received antibiotics or other medicines, vitamins or other treatments that would interfere with the inoculation and propagation of target pathogens on the exterior of the carcass. The resulting calf carcass models the tissues on an adult bovine carcass and provides a medium on which an implanted pathogen will propagate unimpeded. The carcass will also accurately reflect the interaction of an antimicrobial with the pathogen.

### BRIEF DESCRIPTION OF THE DRAWINGS

FIG. 1 is a chart showing the effects of different sprays on Salmonella (by treatment);

FIG. 2 is a chart showing the effects of different sprays on Salmonella (by time after application);

FIG. 3 is a chart showing the effects of different sprays on E. coli O157:H7 (by treatment);

FIG. 4 is a chart showing the effects of different sprays on E. coli O157:H7 (by time after application);

FIG. 5 is a chart showing Salmonella results (Least Square Means);

FIG. 6 is a chart showing Salmonella results (Least Square Means); and

FIG. 7 is a chart showing E. coli O157:H7 results (overall estimated LSM).

### DETAILED DESCRIPTION OF THE INVENTION

Various embodiments are described more fully below and in the referenced example which forms a part hereof, and which show specific embodiments for practicing the invention. However, embodiments may be implemented in many different forms and should not be construed as limited to the embodiments set forth herein.

The invention is applicable to the testing and validation of an antimicrobial's efficacy on beef carcasses. As used herein, “beef” generally refers to the meat of domesticated cattle. Cattle are the most common type of large domesticated ungulates. They are a prominent modern member of the subfamily Bovinae, the most widespread species of the genus Bos, and are most commonly classified collectively as Bos primigenius. Despite the focus on “beef” in this application, the concepts described herein may also be useful in replicating antimicrobial efficacy on other large animals, e.g., buffalo, deer, elk, etc.

It is desirable to accurately ascertain the efficacy of antimicrobials as a “carcass spray” or dip. As a carcass spray, an antimicrobial can be applied early in the processing of the beef so that pathogens resident on the carcass are eliminated and deterred from regrowth. Antimicrobial intervention at this stage also mitigates the possibility that additional colonies of pathogen will be effectively established by subsequent contact with other contaminated beef, equipment and surroundings or via the environment, e.g., airborne bacteria.

For the reasons noted previously, it is not possible to verify and assess the efficacy of an antimicrobial agent on beef carcasses in a meat packing plant by actually using the agent as an intervention on the processing line. For food safety reasons, it is not permissible to inoculate the production carcasses with pathogens or to introduce the pathogens into the meat production facility. In addition, use of an antimicrobial agent on carcasses on a production line does not enable quantification of the agent's efficacy. Because the degree of contamination inherent on the untreated carcasses is unknown, it is impossible to calculate the efficacy or relative efficacy of an antimicrobial agent by observing pathogen counts on the treated beef. At best, one can get a “feel” for efficacy of the antimicrobial agent by comparing bacteria counts on a number of carcasses over a period of time using standard quality control procedures.

Thus, it is customary to initially evaluate the efficacy of a new antimicrobial agent against a target pathogen using in vitro studies. In other words, the antimicrobial is tested against the pathogen on an artificial substrate, i.e., “agar,” in a laboratory. The efficacy of the antimicrobial agent is measured by the reduction (i.e., “log kill”) of the pathogen colony as a result of the antimicrobial intervention. If successful or at least promising, the antimicrobial agent can be further evaluated in “bench top” tests using beef cuts inoculated with the target pathogen. Again, the efficacy can be observed and calculated as the “log kill” reduction in the pathogen colony as a result of the antimicrobial treatment.

These initial evaluations currently form the basis for regulators and beef producers to make decisions as to whether an antimicrobial treatment can be used effectively and safely in beef production. Usually, approval is granted for use at one or a few production plants. Later, approval may then be extended for wider use under specified constraints regarding, for example, the method of application and the concentration of the antimicrobial.

Results of using an antimicrobial in a production plant may vary from the initial laboratory evaluations. Because the tissue composition of beef cuts is different from that of carcasses and because individual beef cuts may vary depending on the health and medical history of the original animal, the correlation in results between beef cut tests and application on carcasses is imperfect. Moreover, it is difficult to assess efficacy of the antimicrobial in actual beef production, since the extent of contamination on any carcass is unknown and, therefore, the “log kill” cannot be observed and calculated routinely.

Thus, a process of accurately assessing and verifying the efficacy of an antimicrobial as a beef carcass spray on a “log kill” basis would be very helpful. In particular, it is desirable to provide both regulators and beef producers with more reliable information on which to base their decisions regarding antimicrobial use as a carcass spray in a meat packing plant.

Young cattle are generally referred to as “calves”; the term “calf” referring to the young animal from birth to weaning (about 9 months). In some instances, the term “calf” may be used until the animal is a yearling. Calves weighing less than about 730 lbs. (i.e., a typical weight at about eight to nine months of age) are slaughtered for “veal” and for other edible products, e.g., calf's liver.

Colostrum is a form of milk produced by the mammary glands of cattle and other mammals in late pregnancy, e.g., within a day or two of calving. Colostrum is very rich in proteins and vitamins and also contains significant growth factors and antimicrobial factors. The antibodies in colostrum provide passive immunity. They are passed to the neonate and provide the first protection against pathogens. The passive immunity from the mother gets transferred to the newborn calf and normally would be maintained and/or enhanced by continued nursing of the growing calf. Nearly every tissue of a newborn calf contains antibodies against many pathogens that a calf would normally encounter. Typically, the calves that are to be retained, i.e., heifers and a few bull calves, are fed colostrum for some period of time as they mature. The majority of bull calves are not fed colostrum, since a large number of them are consigned for veal production.

Calves useful for the present invention are those that do not have significant retained pre-natal immunity from their cows and have not been fed colostrum to replenish that immunity post-birth. The calves should be small enough that their carcasses are of a manageable size in a laboratory.

Calves that have these characteristics typically weight about 50 to 100 pounds and are about 4-10 days old. They have not been fed colostrum post-birth. Accordingly, immunity from the mother should have dissipated substantially, and the immunity has not been renewed with additional colostrum. These animals may be fed colostrum-free milk from other cattle, powdered milk mixed with water, and/or dry calf food.

In a particular embodiment of the present invention, the calf to be used has been denied colostrum since birth. At the very least, the calf has not ingested colostrum recently before slaughter and does not have a special resistance to pathogens such as E. coli or Salmonella normally imparted by colostrum. The calf is denied colostrum by physically separating the calf and its cow or by using nose rings or nosebands on the calf which cause a cow to reject the calf as it attempts to nurse. The calf is not fed colostrum from any other source.

In embodiments of the present invention, the calf should also be denied antibiotics or other medicines, vitamins or other treatments that would interfere with the inoculation and propagation of target pathogens on or in the calf carcass. For example, care should be taken that the calf is not fed commercial calf food that inherently contains antibiotics.

As noted above, in one embodiment of the present invention calves are used that are about four to ten days old. Such animals have muscle and sinew tissues similar to an adult bovine. Although the calves do not have the same thick layer of surface fat (particularly on the belly and back) as an adult animal, in general the calves have enough fat on their carcass to serve as an accurate test model. Larger, older, calves with more surface fat could be used as test models, but the cost of raising them and the difficulties of handling larger animals in the laboratory make them less desirable as test specimens.

A calf identified as a test specimen is slaughtered at the appropriate time, skinned, dismembered and eviscerated. The resulting calf carcass is then inoculated with a target pathogen, e.g., E. coli, by spraying, dipping or swapping. In a preferred embodiment, the entire carcass is inoculated with the target pathogen. Subsequently, the carcass is swabbed with one or more antimicrobial agents at various places—preferably those with differing tissue compositions. For example, those places could include: the surface of the carcass, portions that are the source of round and/or chuck cuts, rib(s), the inside of the rib cage, etc. The places where the antimicrobial has been applied are marked. Samples are subsequently taken from those places, and the effectiveness of the antimicrobial in combatting the target pathogen is measured.

In one embodiment of the present invention the antimicrobial is swabbed on portions of both the fore-quarters and hind-quarters of the carcass (normally the break between the 12^{th} and 13^{th} ribs on an adult bovine carcass) so that effectiveness of the antimicrobial on both portions of the animal is measured.

The following is a more specific example of a particular test procedure applied to calf carcasses that have not received colostrum at any time between birth and slaughter. The general sequence of steps was as follows:

1. Carcasses were washed with hot water. (No antimicrobial was applied.)

2. Carcasses were transported to a secure pathogen handling facility.

3. Carcasses were suspended by the hind limb from a suitable frame.

4. Carcasses were inoculated by bathing them in a suspension of the pathogenic organism.

5. Carcasses were re-suspended from the frame and 30 minutes allowed for the organisms to attach.

6. Swabs were taken from various places to ascertain the level of “contamination.”

7. Carcasses were sprayed with the antimicrobial under investigation.

8. Microbiological samples were collected using swabs from the surface of the carcass.

Individual results from these tests indicate the efficacy of the antimicrobial against the target pathogen on specific beef tissues, and the results collectively indicate the efficacy of the antimicrobial on the entire carcass. The variety of tissues presented in a single test specimen (i.e., calf carcass) give a better indication of anticipated effect of the antimicrobial as an intervention on an adult bovine carcass. The absence of pre-natal and post-natal immunity (from colostrum) in the test specimen also gives a more accurate assessment of the true effect of the antimicrobial agent unbiased by the effects of antibodies in the calf carcass. The same reliability cannot be obtained from attempting to assess an antimicrobial's efficacy using beef “cuts.” The small size of the calf carcass makes it relatively easy to work with as a test specimen in the lab.

### Example

Four, intact male dairy calves age 7 to 10 days were used as test models. The calves had not been fed colostrum. The calves were purchased and transported to the Texas Tech University G.W. Davis Meat and harvested under humane and sanitary conditions using procedures common to the beef processing industry. Once the harvest process is complete, the hot carcasses were transported to the Pathogen Processing Laboratory located on the Texas Tech University campus for antimicrobial testing.

Tests were performed to evaluate “BEEFXIDE®” as an antimicrobial on adult beef carcasses. The antimicrobial used in the tests is a mixture of lactic and citric acids manufactured by Purac America, Inc., Lincolnshire, Ill., under the designation “CL 21/80” and sold under the trademark “BEEFXIDE®” by Birko Corporation, Henderson, Colorado.

### Methodology

### Calves

Four, intact male dairy calves aged 7 to 10 days will be purchased from a dairy farm within 45 miles of Lubbock, Tex. The calves will be purchased and transported to the Texas Tech University G.W. Davies Meat Laboratory and harvested under humane and sanitary conditions using procedures common to the beef processing industry. Once the harvest process is complete, the hot carcasses will be transported to the Pathogen Processing Laboratory (<1 mile) located on the Texas Tech University campus for microbial testing.

### Cocktail Preparation

An inoculum mixture of four E. coli O157:H7 strains (A4 966, A5 528, A1 920 and 966), all originally isolated from cattle, and three Salmonella strains (Typhimurium ATCC 14028, Heidelberg Sheldon 3347-1, and Enteritidis Phage Type 13) were prepared separately. Individual strains were propaged in trypicase soy broth at 37° F. for 18-24 hours. After growth, a concentrated culture was prepared containing all four strains of E. coli O157:H7 and a separate one containing all three strains of Salmonella (25). These E. coli O157:H7 and Salmonella inoculums were frozen at −80° F. and held until processing day. On the day of processing, one tube of frozen culture was thawed by allowing it to sit on the counter top for five minutes. The culture was vortexed and serial dilutions were performed to produce a 10⁵-10⁶ CFU/ml inoculum tubes. An extra tube of culture was sampled on the day of processing and was verified for initial inoculation load.

### Processing

The carcasses were dipped in the inoculum mixture of E. coli O157:H7 or Salmonella (with the exception of one carcass, which will serve as a negative control), for one minute separately. The inoculated sides will be held for 30 minutes to allow for attachment. The sides will then be randomly assigned to one of the following treatments: 1) negative control (no inoculation or treatment); 2) positive control (inoculated but no treatment); 3) BEEFXIDE®; and 4) sterile water (140-145° F.). BEEFXIDE® treatment will be 2.5 wt % concentrations applied at heated temperature (140-145° F.) and sufficient to completely wet the surface of the carcass. The sterile water and BEEFXIDE® was sprayed by hand on the carcasses' sides using a commercial spraying system for one minute at a 40 P.S.I. Carcasses were sampled at approximately one minute and again at one hour after treatment, sampling a Four-100 cm² area of the carcass using a sterile SPONGESICLE® (Biotrace International Inc., Bothell, Wash.) and sterile templates (USDA-050 template, Biotrace International, Muncie, Ind.).

### Microbial Analysis

The total Salmonella and E. coli O157:H7 present will be determined by plating onto XLD agar and MacKonkey agar, respectively. Agars will have a thin-layer overlay of TSA to allow for injured cell recovery. Plates will be incubated at 37° C. for 48 hours and total numbers of pathogens present on the control samples compared to the treated samples will be compared to determine the effectiveness of the intervention.

### Results

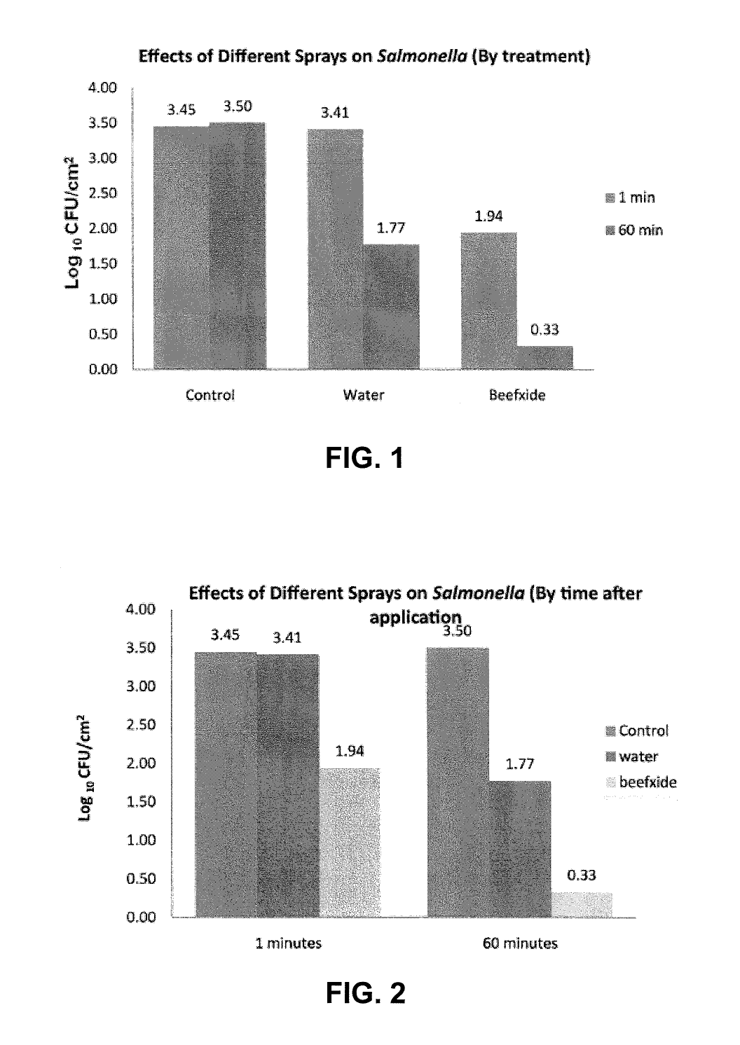

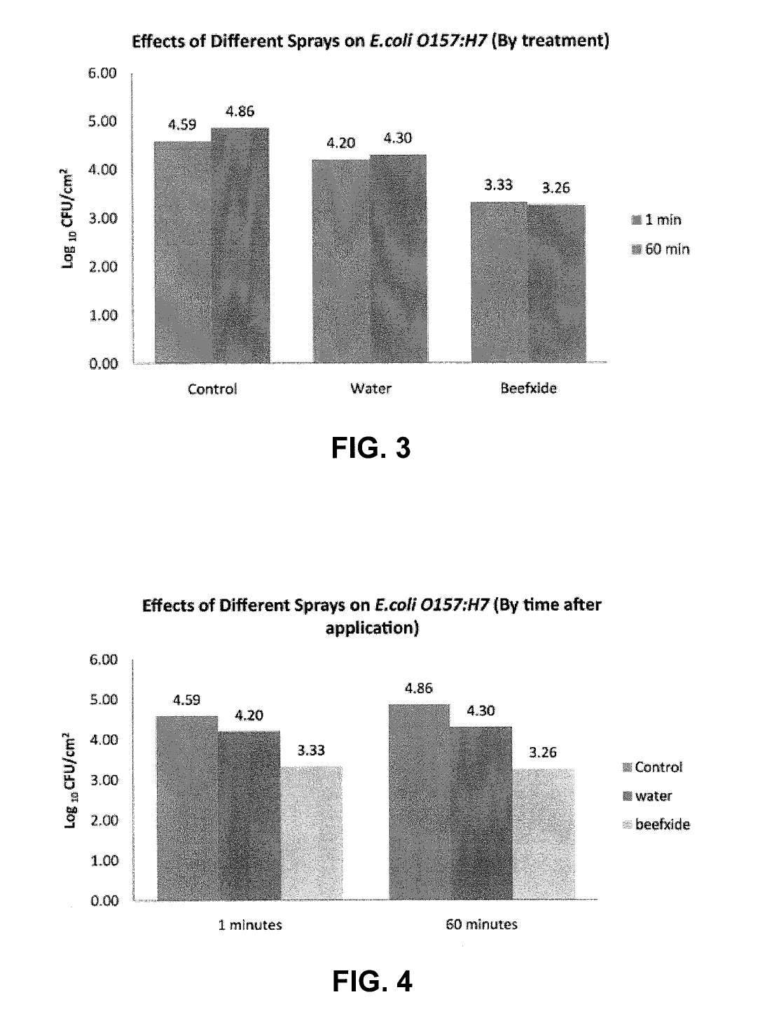

There was no detection of E. coli O157:H7 or Salmonella in the “Natural” samples (used for background flora). The Salmonella and E. coli O157:H7 cocktail were 5.96 log CFU/ml. Salmonella results can be observed in FIGS. 1, 2, 5 and 6. E. coli O157:H7 results can be observed in FIGS. 3, 4 and 7.

TABLE 1
Effects of BEEFXIDE ® applied to inoculated veal carcasses. Values
are expressed in log₁₀ CFU/cm² (based on a total area of 400 cm²/carcass).

| | E. coli O157:H7 | | Salmonella | |
|---|---|---|---|---|
| | 1 minute | 60 minutes | 1 minute | 60 minutes |
| Control | 4.58 | 4.86 | 3.44 | 3.50 |
| Water Spray | 4.41 | 4.27 | 3.39 | 3.62 |
| BEEFXIDE ® | 3.30 | 3.26 | 1.92 | 0.33 |

Because the results are obtained after one replication, statistics could not be accurately run. However, it was decided to use each one of the plates obtained for each of the composites and treat the values as pseudoreplication. The following results were obtained in this way after analysis.

### Salmonella Results:

The F-test was significant (P<0.0001) indicating that there were differences between the treatments. There were also significant differences observed due to time after application (1 vs 60 minutes; P=0.0231). There is also an interaction between the treatment and the time of sampling (P=0.0049) (FIG. 6) (Table 2).

Significant differences were observed between those carcasses treated with BEEFXIDE® and water (P>0.0001) and the BEEFXIDE® and the control (P<0.0001). The water and the control treatments were not significantly different (P=0.8248).

TABLE 2
Type 3 Tests of Fixed Effects

| | Num | Den | | |
|---|---|---|---|---|
| Effect | DF | DF | F Value | Pr > F |
| TRT | 2 | 5 | 136.06 | <.0001 |
| TIME | 1 | 5 | 10.47 | 0.0231 |
| TRT*TIME | 2 | 5 | 18.56 | 0.0049 |

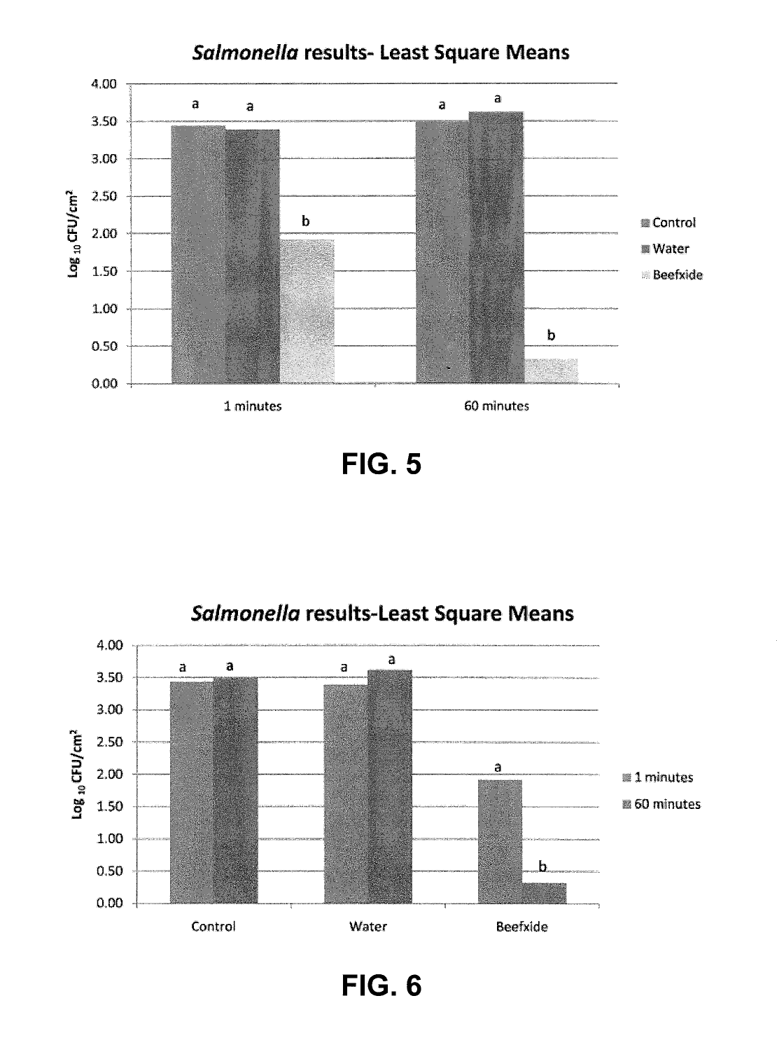

The Least Square Means obtained in this way can be represented as shown in FIG. 5.

E. coli O157:H7 Results:

The F-test was significant (P<0.0001) indicating that there was an overall difference observed between the treatments; however, no significant effects were observed in the levels of E. coli 0157:117 due to time after application (Table 3).

TABLE 3
Type 3 Tests of Fixed Effects

| | Num | Den | | |
|---|---|---|---|---|
| Effect | DF | DF | F Value | Pr > F |
| TRT | 2 | 5 | 100.31 | <0.0001 |
| TIME | 1 | 5 | 0.12 | 0.7438 |
| TRT*TIME | 2 | 5 | 2.18 | 0.2091 |

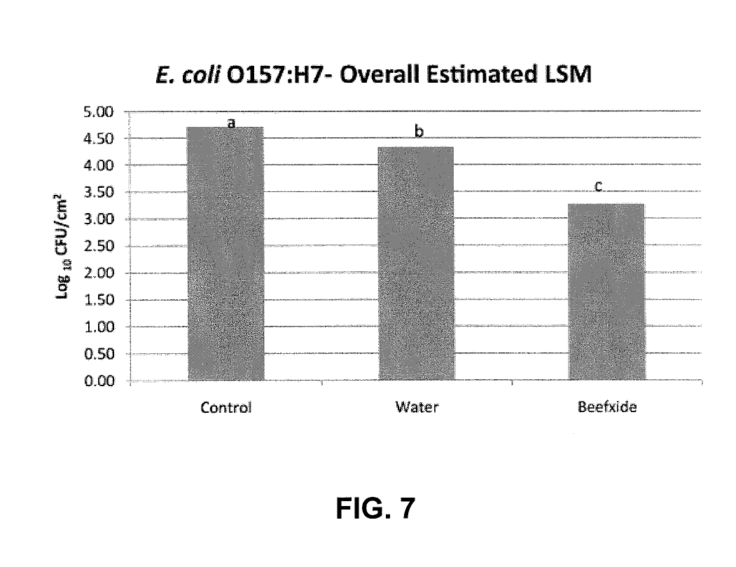

Results obtained in this way indicate that there were differences between BEEFXIDE® and both the control and the water treatments (P<0.0001, P=0.0002, respectively) (FIG. 7); control and water treatments were also significantly different (P=0.0160) (FIG. 7); however, the overall difference between treatments varied between 0.16 and 1.44 logs (FIG. 7).

1. A method for accurately assessing and verifying the efficacy of an antimicrobial agent as a bovine carcass spray:
providing a test specimen comprising at least one carcass from a bovine calf an animal that has not received colostrum after birth;
applying a pathogen to the specimen at multiple places;
applying an antimicrobial agent to the target pathogen on the specimen; and
observing the effect of the antimicrobial agent on the pathogen.

2. The method of claim 1, wherein the test specimen comprises beef.

3. The method of claim 1, wherein the antimicrobial agent is applied as a carcass spray.

4. The method of claim 1, wherein the antimicrobial agent is applied as a dip.

5. The method of claim 1, wherein providing a test specimen comprising at least one carcass from an animal that has not received colostrum after birth comprises denying colostrums to the animal by physically separating the animal from its mother.

6. The method of claim 1, wherein providing a test specimen comprising at least one carcass from an animal that has not received colostrum after birth comprises using nose rings or nosebands on the animal to prevent nursing of the animal.

7. The method of claim 1, wherein the animal has not been exposed to antibiotics, medicines, or vitamins.

8. The method of claim 1, wherein the animal has not been fed commercial animal food.

9. The method of claim 1, wherein the animal is about four to ten days old.

10. The method of claim 1, wherein applying a pathogen to the specimen comprises spraying the pathogen on the specimen.

11. The method of claim 1, wherein applying a pathogen to the specimen comprises inoculating the specimen by dipping the specimen in a fluid containing the pathogen.

12. The method of claim 1, wherein applying a pathogen to the specimen comprises applying E. coli on the specimen.

13. The method of claim 1, wherein applying a pathogen to the specimen comprises applying the pathogen on the entire specimen.

14. The method of claim 1, wherein applying the antimicrobial agent to the target pathogen on the specimen comprises applying a plurality of antimicrobial agents to the target pathogen.

15. The method of claim 1, wherein applying the antimicrobial agent to the target pathogen on the specimen comprises applying a plurality of antimicrobial agents to the target pathogen.

16. The method of claim 15, wherein applying a plurality of antimicrobial agents to the target pathogen comprises applying the plurality of antimicrobial agents on differing tissue compositions on the specimen.

17. The method of claim 1, wherein applying the antimicrobial agent to the target pathogen on the specimen comprises applying the antimicrobial agent to fore-quarters and hind-quarters of the target pathogen.

18. A target specimen for assessing and verifying the efficacy of an antimicrobial agent as a bovine carcass spray comprising at least one carcass from a bovine calf that has not received colostrum after birth.

19. The target specimen of claim 18, wherein the bovine calf has not been exposed to antibiotics, medicines, or vitamins.

20. The method of claim 1, wherein the bovine calf has not been fed commercial animal food.
